(19) United States
(12) Patent Application Publication
(10) Pub. No.: US 2025/0152523 A1
(43) Pub. Date: May 15, 2025

(54) COMPOSITIONS FOR TREATMENT OF SEXUAL DYSFUNCTION

(71) Applicant: Woody's Medical LLC, Scottsdale, AZ (US)

(72) Inventors: Austin Lucht, Scottsdale, AZ (US); Suzanne Bentz, Scottsdale, AZ (US)

(73) Assignee: Woody's Medical LLC, Scottsdale, AZ (US)

(21) Appl. No.: 18/505,224

(22) Filed: Nov. 9, 2023

(51) Int. Cl. A61K 31/135; A61K 9/48; A61K 47/38

(52) U.S. Cl. CPC A61K 31/135; A61K 9/4866; A61K 47/38

(57) ABSTRACT

In various embodiments compositions for the treatment of sexual dysfunction, particularly premature ejaculation, comprise at least one selective serotonin reuptake inhibitor (an SRRI) and a carrier encased in a rapidly disintegrating immediate release capsule shell. In preferred embodiments, the carrier comprises microcrystalline cellulose that functions as both bulk filler and disintegrant. The PE compositions herein are administered in capsule dosage form as needed rather than continuously, thus drastically reducing the risk for long term health issues seen with chronic daily SRRI regimens.

### FIELD

This disclosure generally relates to pharmaceutical compositions, and more specifically, to orally administered compositions usable to treat male premature ejaculation.

### BACKGROUND

Premature ejaculation is defined as male penile ejaculation in response to only minimal sexual stimulation and remains one of the most common complaints men have regarding sexual performance. Ejaculation that occurs sooner than desired can be mentally humiliating for a man and can lead to other sexual dysfunctions including erectile dysfunction (ED), low sexual desire, and even an aversion to sexual intimacy. See, for example, J. Rust J., et al., British Journal of Psychiatry, 152 (5), 629-631 (1988).

Over twenty years ago certain selective serotonin reuptake inhibitors (SSRIs) were disclosed in methods for the prevention, treatment, and management of sexual disfunction. See, for example, PCT Application Publication WO2001017521 filed by Eli Lilly and Company. Unfortunately, SSRIs have proven to require constant medical follow up and oversight to ensure patient safety and proper outcomes.

Further, medications presently available to treat premature ejaculation based on SSRIs are provided as orally dosed tablets that need to be taken chronically over an indefinite timespan, rather than taken just once prior to planned sexual contact. This chronic prescription is impractical and can lead to serious long-term issues such as sexual dysfunction, weight gain, sleep disturbances, coronary heart disease, and cardiovascular mortality.

In view of these and other serious shortcomings, new dosage forms and treatment regimens are still needed for management of premature ejaculation in male patients.

### SUMMARY

It has now been surprisingly discovered that SRRIs, particularly dapoxetine, are more effective for treatment of premature ejaculation when administered as needed in instant delivery (ID) capsules. This finding is contrary to prior teaching that SRRIs like dapoxetine must be administered as slow release orally ingested tablets in a regimen prescribing chronic daily administration. SRRIs like dapoxetine have not been previously compounded or dosed in the form of ID capsules.

In various embodiments, new compositions for the treatment of premature ejaculation (or more simply, “PE compositions”) are described. In various embodiments, PE compositions are compounded in a dosage form for oral administration and rapid disintegration and release.

In various embodiments, new PE compositions comprise an immediate release oral dosage form.

In various embodiments, new PE compositions comprise an immediate release capsule oral dosage form.

In various embodiments, a pharmaceutical dosage form for oral administration of a PE composition comprises a capsule and a PE composition comprising at least one SRRI and a filler, optionally an excipient, enclosed within the capsule.

In various embodiments, a method of treating premature ejaculation (PE) is disclosed.

In various embodiments of the method, the individual in need thereof is diagnosed as experiencing premature ejaculation or any one of the related indications of retarded ejaculation, low sexual desire, sexual aversion, or inhibited orgasm.

In various embodiments, a pharmaceutical composition for treatment of a male sexual dysfunction, said composition comprises at least one selective serotonin reuptake inhibitor (SRRI): a carrier; and optionally, an excipient: wherein the composition is in the form of a loose dry powder amenable to filling into individual dosage capsules.

In various embodiments, the selective serotonin reuptake inhibitor (SRRI) is selected from the group consisting of dapoxetine, citalopram, escitalopram, fluoxetine, paroxetine, sertraline, pharmaceutically acceptable salts thereof, and mixtures thereof.

In various embodiments, a pharmaceutical dosage form for oral administration comprises a hard-shell capsule; and a composition comprising at least one selective serotonin reuptake inhibitor (SRRI): a carrier; and optionally, an excipient enclosed therein.

In various embodiments, the carrier comprises microcrystalline cellulose.

In various embodiments, the optional excipient is at least one of a disintegrant, a colorant, a flavoring agent, a sweetener, a preservative, and a stabilizer.

In various embodiments, a pharmaceutical composition consists essentially of dapoxetine-hydrochloride and microcrystalline cellulose.

In various embodiments, a single dose pharmaceutical capsule consists essentially of from about 100 mg to about 300 mg of a composition consisting essentially of from about 15 wt. % to about 25 wt. % dapoxetine-hydrochloride and from about 75 wt. % to about 85 wt. % microcrystalline cellulose, based on the total weight of the composition; and a hard-shell capsule enclosing said composition.

In various embodiments, the capsule comprises gelatin or vegetarian hydroxypropyl methylcellulose (HPMC), and wherein the capsule is optionally laser-perforated with a plurality of holes for water ingress.

In various embodiments, each capsule provides about 15 mg to about 60 mg dapoxetine.

In various embodiments, a method of treating a sexual dysfunction in a male individual comprises orally administering to the individual in need thereof a therapeutically effective amount of a loose powder composition packaged in an immediate delivery capsule dosage form, said composition comprising at least one selective serotonin reuptake inhibitor (SRRI): a carrier; and optionally, an excipient, wherein the SRRI is selected from the group consisting of dapoxetine, citalopram, escitalopram, fluoxetine, paroxetine, sertraline, pharmaceutically acceptable salts thereof, and mixtures thereof.

In various embodiments, the sexual dysfunction is characterized by premature ejaculation, retarded ejaculation, low sexual desire, sexual aversion, or inhibited orgasm.

In various embodiments, the therapeutically effective amount comprises orally administering from about 15 mg to about 60 mg of the at least one SRRI as needed and within 3-hours prior to an expected sexual encounter.

In various embodiments, the at least one SRRI is dapoxetine and the carrier is microcrystalline cellulose.

In various embodiments, the therapeutically effective amount comprises about 138 mg of said loose powder composition.

In various embodiments, the therapeutically effective amount comprises from about 15 mg to about 60 mg of dapoxetine.

### DETAILED DESCRIPTION

The detailed description of exemplary embodiments refers to the accompanying drawings, which show exemplary embodiments by way of illustration and best mode. While these exemplary embodiments are described in enough detail to enable those skilled in the art to practice the invention, other embodiments may be realized, and logical, chemical, and mechanical changes may be made without departing from the spirit and scope of the inventions. Thus, the detailed description is presented for purposes of illustration only and not of limitation. For example, unless otherwise noted, the steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

Compositions for the treatment of premature ejaculation (PE) comprising a selective serotonin reuptake inhibitor (or “SSRI”) are described. In various embodiments, PE composition in accordance with the present disclosure comprises at least one SSRI, a filler, and optionally at least one excipient. In various embodiments, a filler includes a disintegrant or it exhibits properties of a disintegrant while providing bulk volume and weight to a composition.

### Definitions

As used herein, the term “premature ejaculation” collectively refers to number of male sexual dysfunctional issues including, but not limited to, persistent or recurrent ejaculation with minimal sexual stimulation before, upon, or shortly after penetration, retarded ejaculation, low sexual desire, sexual aversion, and inhibited orgasm.

As used herein, the acronym “SSRI” refers to “selective serotonin reuptake inhibitors,” which are a class of drugs typically used as antidepressants. SSRIs are believed to increase extracellular levels of serotonin by limiting reabsorption into presynaptic cells. SSRIs are also prescribed for anxiety disorders, such as social anxiety disorder, generalized anxiety, panic disorder, obsessive compulsive disorder (OCD), eating disorders, chronic pain and PTSD. For treatment of premature ejaculation, SSRIs are not administered prior to sexual activity, but instead are taken on a chronic daily basis, as this regimen has been found more effective. See, for example, M. D. Waldinger, “Premature Ejaculation: State of the Art,” The Urologic Clinics of North America, 34 (4), 591-599, (2007). Some of the known SRRIs finding use herein include, but are not limited to, dapoxetine, citalopram, escitalopram, fluoxetine, paroxetine, and sertraline. Each of these SRRIs may be in the form of pharmaceutically acceptable salts, solvates, or hydrates.

As used herein, the term “dapoxetine” refers to the pharmaceutical active having the chemical structure according to Formula (I):

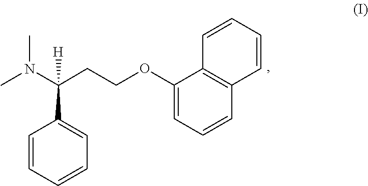

with IUPAC name(S)-N,N-dimethyl-3-(naphthalen-1-yloxy)-1-phenylpropan-1-amine, CAS No. 119356-77-3 and MW 305.41 g/mol. In various embodiments, dapoxetine salts and prodrugs thereof may be used in various compositions herein, such as for example, dapoxetine-HCl (CAS No. 129938-20-1, MW 341.9 g/mol).

As used herein, the term “immediate release” or “immediate delivery” or “instant delivery” or “ID” refer to a pharmaceutical dosage form designed to fall apart, dissolve, or otherwise disintegrate as quickly as possible. Typically, a dosage form for immediate release will be an oral dosage form, such as a tablet for sublingual drug administration or a capsule for swallowing with subsequent GI drug delivery. Capsule dosage forms may be designed for immediate drug release by physical or chemical changes to either the capsule shell, or the powder fill composition, or both. For example, hard capsule shells may be laser-drilled with holes that allow entry of water in the gut. In other examples, vegetarian cellulose capsules, such as hydroxypropyl methylcellulose (HPMC capsules), may be preferred over gelatin capsules in view that gelatin polymers tend to be crosslinked which reduces dissolution rates. In various examples, one or more disintegrating agents (called “disintegrants”) may be added into the powder fill composition of a capsule, or into the capsule shell itself to accelerate physical deterioration. In various embodiments, immediate release may also be coupled with increased GI absorption, and in those instances an intestinal permeation enhancer may be included in an immediate release composition so that there is not only rapid dissolution of the capsule but also intestinal absorption. In various embodiments, a standard gel cap or powder filled hard gelatin capsule may provide immediate release in the gut simply in response to physiological conditions of heat and acid in the stomach, sufficiently capable of dissolving a gelatin shell in due course. Immediate release tends to be the opposite of controlled release or sustained release.

As used herein, the term “carrier” refers to a material or mixture of materials that are not pharmacologically active, and that may be added in quantity sufficient to complete a formula to “100 wt. %” total. In various embodiments, PE compositions herein comprise at least one SRRI and optional excipient, with the remainder comprising a carrier. Carriers increase weight and may be seen to convey pharmaceutical actives to a particular locale, but typically do not contribute to any pharmacological effect. Pharmaceutically acceptable carriers are known to the pharmaceutical arts, and include such substances as monosaccharides and disaccharides, (mannitol, lactose, dextrose), cellulosic substances (microcrystalline cellulose MCC), carbonates (calcium carbonate), phosphates, (calcium phosphate), and sulfates (calcium sulfate). For an exhaustive listing of pharmaceutically acceptable carriers, see “Handbook of Pharmaceutical Excipients, 6th Edition, R. C Rowe, et al., editors, Pharmaceutical Press, London, 2009. Microcrystalline cellulose has properties of a disintegrant and thus can be used as both disintegrant and carrier. MCC enhances drug dissolution by expanding in size when in contact with water, causing tablets and capsules to break apart faster in the GI tract. In other embodiments, a PE composition herein may include a separate disintegrant as an excipient with a carrier.

As used herein, the term “excipient” refers to additional functional ingredients in a PE composition, and thus distinct from inert filler. Excipient refers to those ingredients that although functional, play a minor role in the composition and no pharmacological role. These ingredients typically include disintegrants, colors, flavorants, sweenteners, and preservatives.

As used herein, the term “composition” takes on the ordinary meaning in formulation chemistry as a combination of ingredients. In various embodiments, a composition is designed to adopt a particular physical form, or at least be amenable to physical change into a desired physical form, which may be the dosage form for a particular treatment regimen. Typically, a composition is made homogeneous by mixing or blending, although not all liquid compositions are colorless and transparent and not all powder compositions are white and perfectly powdered or granular. Compositions comprising an emulsion, dispersion or suspension may be homogeneous because the droplets or particles are evenly spread in a carrier. So, for example, a composition herein may be in the form of a thin liquid (having a viscosity at or near that of water), a viscous liquid (having a liquid of viscosity greater than water), a paste, a cream, a jelly, a gel, or a powder. Ingredients for a composition herein are generally shown “as added,” meaning there is a possibility for one or more chemical reactions between ingredients once the ingredients are mixed together, such as into a common carrier. One skilled in the art of formulation chemistry can recognize whether ingredients might react in a mixture. These reactions can include neutralization (e.g., between acid and alkali ingredients), mixed micelle formation (mixed surfactants in liquid systems) or other encapsulation phenomena, hydrolysis, and so forth. In various embodiments, a composition herein comprises a blended powder that can be packed into capsules for oral administration. In some instances, a composition may take into consideration an outer encasing when that material is also included in the administration of the composition to an individual. For example, a gelatin capsule may be included in the listing of ingredients for a composition, or perhaps just the ingredients of the contents of the capsule may be listed. In various embodiments, ingredients in a composition are listed in “weight percent,” (i.e., “wt. %”), based on the total weight of the composition. For example, 100 milligrams of a composition comprising 40 mg A and 60 mg B may be recited as “40 wt. % A and 60 wt. % B. based on the total weight of the composition,” which necessarily totals to “100 wt. %.” The actual weight amounts, (e.g., milligrams or grams) generally refers to amounts added for a particular batch size, (e.g., a batch size of powder usable to fill 100 capsules).

As used herein, the term “dosage form” takes on its ordinary meaning in the pharmaceutical arts as the physical form of a composition designed for a particular administration route. For example, dosage forms include, but are not limited to, injectables, infusible liquids, nasal sprays, nasal gels, topicals such as transdermal creams, ointments and patches, loose powders, tablets, sublingual tabs, capsules, lozenges, syrups, vapors, and so forth. In various embodiments, compositions of interest herein comprise powders, and the dosage form preferably comprises a capsule comprising the powdered composition enclosed, encased or “encapsulated” in the capsule or a tablet comprising the powdered composition compressed into a shape for swallowing or sublingual dissolution.

As used herein, the term “capsule” generally refers to a one or two-piece enclosure for a loose powder, which can be swallowed for oral administration of the powder contents of the capsule, or a soft-shell for a liquid composition, otherwise known as a “gel cap”. In general, “soft” capsules for liquids are one piece, whereas “hard” capsules for powders are two-piece. In some instances, the capsule is said to “encapsulate” the powder contained therein, which can be confusing because the term “encapsulation” is often used, perhaps more correctly, to describe a microscale or nanoscale phenomenon rather than describing something macroscopic like a drug dosage form. In various embodiments, capsules for use herein are hard, stable two-piece shells, or enclosures, capable of stably holding a powder fill, and capable of disintegrating in the gastrointestinal track of an individual. Capsules for use herein may comprise any combination of animal gelatin, plant polysaccharides (carrageenan, cellulosic substances, etc.), or starch or derivatives thereof. In some instances, capsules may further comprise plasticizers, colors, preservatives, disintegrants, lubricants, and various surface treatments such as laser perforations. In various embodiments, a capsule may be transparent so that the contents are visible, or entirely opaque to obscure the contents. In various embodiments, the rate of delivery of an SRRI from a two-piece hard capsule having a powder fill may be controlled by any combination of ingredients in the powder fill and ingredients or design configurations of the capsule itself. For example, a capsule intended for immediate release of an active drug may comprise a micronized powder fill in combination with a laser perforated capsule having disintegrants incorporated in the capsule material.

Two-piece hard capsules for use herein can be characterized by a size scale that includes size 5, 4, 3, 2, 1, 0, OE, 00, 000, 13, 12, 12el, 11, 10, 7 and Su07, (in increasing order of physical dimensions and internal volume when assembled). Typically, only the capsule sizes from #5 up through about 000 (26.14 mm×9.91 mm, 1.36 mL) would be practical for human oral consumption, and digestive tract (enteral) route of administration. Preferred for use herein are #3 size capsules. A #3 capsule shell weighs about 50 mg if made from vegetarian HPMC and about 48 mg if made from gelatin. As mentioned, and although somewhat more expensive, the vegetarian HPMC shell is preferred because of its faster dissolution rate compared to gelatin. In preferred embodiments, a fill weight for an individual rapid release capsule is from about 100 mg to about 300 mg of loose powder.

As used herein, the term “subject” or the phrase “a subject in need thereof” refers to any human or non-human animal requiring or desirous of a pharmacological change. For example, a subject in need thereof may be a male human patient clinically diagnosed with a sexual dysfunction such as premature ejaculation. In various embodiments, the subject in need thereof is a person desirous of improved sexual performance as subjectively perceived by that individual. Most importantly, a subject in need thereof can be any human in good health, but desirous of heightened sexual experiences.

As used herein, the term “treatment” of a subject (e.g., a human) is any type of intervention used in an attempt to alter the natural course of the subject. Treatment includes, but is not limited to, administration of a PE composition in accordance with the present disclosure and may be performed prior to a sexual encounter.

As used herein, the term “therapeutically effective amount” refers to a minimum dosage of a composition in accordance with the present disclosure that provides a desired effect. Therefore, a therapeutically effective amount varies by subject, dosage form, concentration of one or more SRRIs in the PE composition, and the ultimate results desired. For example, a therapeutically effective amount of a capsule disclosed herein to treat an individual experiencing PE might be one (1) to two (2) capsules ingested just prior to an anticipated sexual encounter, wherein each capsule weighs from about 100 mg to about 150 mg and delivers from about 15 mg to about 60 mg total active SRRI per capsule.

As used herein, the term “modulate” includes to “increase” or “decrease” one or more quantifiable parameters, optionally by a defined and/or statistically significant amount. By “increase” or “increasing.” “enhance” or “enhancing,” or “stimulate” or “stimulating.” refers generally to the ability of one or more PE compositions in accordance with the present disclosure to produce or cause a greater physiological response (i.e., downstream effects) in a cell or in a subject relative to the response caused by either no PE composition or a control compound. Relevant physiological or cellular responses (in vivo or in vitro) upon administration of a PE composition will be apparent to persons skilled in the art. An “increased” or “enhanced” amount is typically a “statistically significant” amount, and may include an increase that is 1.1, 1.2, 2, 3, 4, 5, 6, 7, 8, 9, 10, 15, 20, 30, 40, 50 or more times (e.g., 500, 1000 times), including all integers and decimal points in between and above 1 (e.g., 1.5, 1.6, 1.7, 1.8), the amount produced by no PE composition (the absence of a bioactive agent) or a control compound.

As used herein, the term “approximately” in reference to amounts refers to plus or minus 5% of the value given, such as wt. %. The term “about,” a in reference to amounts refers to plus or minus 10% of the value given, such as wt. %.

### GENERAL EMBODIMENTS

In various embodiments, a PE composition in accordance with the present disclosure comprises at least one SRRI, optional excipient, and a carrier. In various embodiments, the SRRI is selected from the group consisting of dapoxetine, citalopram, escitalopram, fluoxetine, paroxetine, sertraline, pharmaceutically acceptable salts, solvates or hydrates thereof, and combinations thereof. In various embodiments, the carrier comprises a cellulosic, preferably microcrystalline cellulose. In various embodiments, the PE composition is in the physical form of a loose powder for filling capsules, and wherein the dosage form to administer the composition comprises an immediate release capsule.

In these general embodiments of PE compositions, the at least one SRRI is present (at a combined weight if more than one) from about 15.0 wt. % to about 25.0 wt. %, based on the total weight of the composition, and depending on the actual SRRI chemical species used and the desired strength of the SRRI in the finished dosage form. In various embodiments, a SRRI may be in the form of a pharmaceutically acceptable salt, such as the hydrochloride salt or other salt to provide a more water soluble and/or absorbable form.

Table 1 sets forth three general embodiments (I, II, III) in accordance with the present disclosure, which vary as to the nature of the SRRI and whether a single SRRI or combination thereof is used. The compositions are in the physical form of loose powders that can be loaded into capsules to provide dosage forms for oral administration. General embodiments I-III are compositions for use in an immediate release dosage form. In any of the specific compositions under I, II, and III, the loose fill composition may be loaded into two-piece hard shell capsules at a fill level of from about 100 mg to about 300 mg, preferably from about 120 mg to about 156 mg, depending on the nature of the one or more SSRI actives. The capsule shell may be selected and/or physically modified as necessary to be more appropriate for immediate decomposition and release.

TABLE 1
General SSRI Compositions for the Treatment of PE

| | Compositions | | |
|---|---|---|---|
| Ingredient (wt. %) | I | II | III |
| SRRI¹ | 15-18 | 18-22 | 22-25 |
| Carrier² | 82 to 85 | 78 to 82 | 75 to 78 |
| Excipients³ (color, flavor, etc.) | 0 to 5 | 0 to 5 | 0 to 5 |
| Total | 100.00 wt. % | 100.00 wt. % | 100.00 wt. % |
| Physical Appearance | Loose powder for | Loose powder for | Loose powder for |
| | capsule fill | capsule fill | capsule fill |
| Typical Capsule Fill | 100 mg to 300 mg | 100 mg to 300 mg | 100 mg to 300 mg |
| Use | Immediate Release | Immediate Release | Immediate Release |

### NOTES to TABLE 1:

(1) The SRRI is selected from the group consisting of dapoxetine, citalopram, escitalopram, fluoxetine, paroxetine, sertraline and mixtures thereof, including pharmaceutically acceptable salts, solvates and hydrates thereof.

(2) In preferred embodiments, the carrier is microcrystalline cellulose (MCC), which also provides a disintegrating effect upon moisture absorption and swelling, negating the need for a separate disintegrant.

(3) Typically the optional excipient includes disintegrant, colors, flavoring agents, sweeteners, preservatives, stabilizers, and so forth. In various embodiments, the flavoring agents are not included, but nonetheless a color, such as a food coloring, may be added for a visual effect when using a transparent and colorless capsule shell. In certain embodiments, the filler is MCC and the need for a separate disintegrant is eliminated.

In various embodiments, a PE composition in accordance with the present disclosure comprises any combination of optional excipients. In various embodiments, a PE composition contains no excipients. In some instances, there is no taste to mask in a swallowed capsule, and perhaps no need for colorants, sweeteners or preservatives. Depending on the SRRI, and in view the filled capsule is designed for immediate release, it may be necessary to include a flavorant.

In various embodiments, the optional one or more excipients include any one or combination of flavorant, sweetener, buffer (or acidic agent and/or alkali agent), colorant. disintegrant, intestinal permeation enhancer, stabilizer, preservative, or other pharmaceutically acceptable substance. Any of these materials not specifically mentioned herein may be found in “Handbook of Pharmaceutical Excipients, 6^{th} Edition, R. C Rowe, et al., editors, Pharmaceutical Press, London, 2009, mentioned above in the context of “fillers.” For liquid excipients, or excipients that might be better dispersed if provided in solution, the substance may be sprayed into a ribbon blender with a spray bar as a powdered composition is blending. In this way, a dry blended powder is still obtained, even though small amounts of liquid ingredients are absorbed in homogeneously.

Suitable flavorants can include, for example, flavors, such as, natural flavors, artificial flavors, and combinations thereof. Non-limiting examples of flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg. allspice, oil of sage, mace, oil of bitter almonds, and cassia oil. Suitable flavoring agents also include, for example, artificial, natural and synthetic fruit flavors such as vanilla, citrus oils (e.g., lemon, orange, lime, and grapefruit), and fruit essences (e.g., apple, pear, peach, grape, strawberry, raspberry, cherry, plum, pineapple, and apricot), and the like, and combinations thereof.

Other flavorants and fragrant aromatics that may be included individually or in combination include, but are not limited to, anethole, menthol, menthone, menthyl acetate, eucalyptol, borneol, borneol acetate, camphor, 1,8-cineole, cinnamaldehyde, benzaldehyde, citral, thujone, eugenol, limonene, geraniol, citronellol, citronellal, pinene, linalool, thymol, carvone, caryophyllene, linalyl acetate, methyl salicylate, and mixtures thereof. Also, substances that provide scent and flavor include, but are not limited to, 3,3,5-trimethylcyclohexanol, methoxycyclohexanol, benzyl alcohol, anise alcohol, cinnamyl alcohol. β-phenyl ethyl alcohol (2-phenylethanol), cis-3-hexenol, musk xylol, isoeugenol, methyl eugenol, α-amylcinnamic aldehyde, anisaldehyde, n-butyl aldehyde, cumin aldehyde, cyclamen aldehyde, decanal, isobutyl aldehyde, hexyl aldehyde, heptyl aldehyde, n-nonyl aldehyde, nonadienol, hydroxycitronellal, benzaldehyde, methyl nonyl acetaldehyde, dodecanol, α-hexylcinnamic aldehyde, undecenal, heliotropin, vanillin, ethyl vanillin, methyl amyl ketone, methyl β-naphthyl ketone, methyl nonyl ketone, musk ketone, diacetyl, acetyl propionyl, acetyl butyryl, acetophenone, p-methyl acetophenone, ionone, methyl ionone, amyl butyrolactone, diphenyl oxide, methyl phenyl glycidate, γ-nonyl lactone, coumarin, cineole, ethyl methyl phenyl glycidate, methyl formate, isopropyl formate, linalyl formate, ethyl acetate, octyl acetate, methyl acetate, benzyl acetate, butyl propionate, isoamyl acetate, isopropyl isobutyrate, geranyl isovalerate, allyl capronate, butyl heptylate, octyl caprylate octyl, methyl heptynecarboxylate, methine octynecarboxylate, isoacyl caprylate, methyl laurate, ethyl myristate, methyl myristate, ethyl benzoate, benzyl benzoate, methylcarbinylphenyl acetate, isobutyl phenylacetate, methyl cinnamate, cinnamyl cinnamate, ethyl anisate, methyl anthranilate, ethyl pyruvate, ethyl α-butyl butylate, benzyl propionate, butyl acetate, butyl butyrate, p-tert-butylcyclohexyl acetate, cedryl acetate, citronellyl acetate, citronellyl formate, p-cresyl acetate, ethyl butyrate, ethyl caproate, ethyl cinnamate, ethyl phenylacetate, ethylene brassylate, geranyl acetate, geranyl formate, isoamyl salicylate, isoamyl isovalerate, isobornyl acetate, linalyl acetate, methyl anthranilate, methyl dihydrojasmonate, β-phenylethyl acetate, trichloromethylphenyl carbinyl acetate, terpinyl acetate, vetiveryl acetate, and mixtures thereof.

Suitable sweeteners include nutritive carbohydrates such as sucrose, glucose, fructose, glucose, trehalose, galactose, mannitol, sorbitol, xylitol and artificial sweeteners such as saccharin, aspartame, acesulfame K, cyclamates, neotame, sucralose, stevia, and neohesperidin dihydrochalcone (NHDC).

Suitable buffers may comprise one or more acidifying agents or alkaline agents as necessary to neutralize various co-ingredients, form salts of various co-ingredients, and/or achieve a particular pH target for the composition, such as to adjust the local environment in the GI tract as a dosage form dissolves.

Exemplary acidifying agents for use in the present compositions include, but are not limited to, organic acids of any molecular weight and mineral acids (inorganic acids), and mixtures thereof. Organic acids may include mono-carboxylic acids, di-carboxylic acids, or tri-carboxylic acids, and may be saturated or may have any degree of unsaturation. For example, organic acids for use in various embodiments of the composition in accordance to the present disclosure may include, but are not limited to, formic acid, carbonic acid, acetic acid, lactic acid, oxalic acid, propionic acid, valeric acid, enanthic acid, pelargonic acid, butyric acid, lauric acid, docosahexaenoic acid, eicosapentaenoic acid, pyruvic acid, acetoacetic acid, benzoic acid, salicylic acid, aldaric acid, fumaric acid, glutaconic acid, traumatic acid, muconic acid, malonic acid, malic acid, succinic acid, glutaric acid, adipic acid, pimelic acid, suberic acid, azelaic acid, abietic acid, pimaric acid, sebacic acid, phthalic acid, isophthalic acid, terephthalic acid, maleic acid, citric acid, and combinations thereof.

Exemplary alkaline materials include any organic amines, NH₃, alkali metal or alkaline earth hydroxide, any conjugate bases of any organic acids (e.g. R—COO⁻), and any of the salts of carbonic acid, phosphoric acid, nitric acid and sulfuric acid, and any mixtures thereof. For example, alkaline materials for use in various embodiments of the composition in accordance to the present disclosure may include, but are not limited to, NaOH, KOH, NH₃, sodium acetate, sodium succinate, disodium succinate, monosodium citrate, disodium citrate, trisodium citrate, NaH₂PO₄, Na₂HPO₄, Na₃PO₄, KH₂PO₄, K₂HPO₄, K₃PO₄, NaHSO₄, Na₂SO₄, KHSO₄, K₂SO₄, NaHCO₃, Na₂CO₃, KHCO₃, K₂CO, NaH₃P₂O₇, Na₂H₂P₂O₇, Na₃HP₂O₇, Na₄P₂O₇, KH₃P₂O₇, K₂H₂P₂O₇, K₃HP₂O₇, KAP₂O₇, and mixtures thereof. Any of these chemical species may exist as various hydrates when purchased as raw materials for use in the present compositions.

Exemplary colorants include the pharmaceutically acceptable colors used for capsules and tablet dosage forms, such as the US FDA certified colors, dyes and lakes for use in pharmaceutical capsules, tablets and syrups. These acceptable colorants include the inorganic pigments such as titanium dioxide, yellow iron oxide, red iron oxide and black iron oxide, the organic pigments such as D&C Red 36, Red 30 and Red 34, the solvent soluble colors D&C Yellow 11, Yellow 7, Red 27, Red 21, Red 17, Green 6, and Violet 2, and the water soluble colors D&C Green 8, Yellow 10, Yellow 8, Orange 4, Red 22, Red 28, Red 33. Green 5, quinoline yellow, FD&C Yellow 5, Yellow 6, Red 4, Red 40, Red 3, Green 3, Blue 1, Blue 2, and ponceau 4R, carmoisine, amaranth, patent blue V and black PN, and a number of “organic lakes.”

Suitable disintegrants include, but are not limited to, sodium starch glycolate, croscarmellose sodium, microcrystalline cellulose (MCC), and crospovidone. Added in sufficient quantity, a disintegrant may comprise the entirety of the carrier by weight. For a review of disintegrants that find use in the present compositions, see P. M. Desai, “Review of Disintegrants and the Disintegration Phenomena,” J. Pharm. Sci., 105, 2545-2555 (2016).

Suitable intestinal permeation enhancers include, but are not limited to, surfactants that assist bio-absorption, including, for example, fatty acids and/or esters or salts thereof, bile acids and/or salts thereof. Bile acids/salts and fatty acids and their uses are further described in U.S. Pat. No. 6,287,860, which is incorporated herein in its entirety. In some embodiments, the present disclosure provides combinations of absorption enhancers, for example, fatty acids/salts in combination with bile acids/salts. An exemplary combination is the sodium salt of lauric acid, capric acid and ursodeoxycholic acid (UDCA) for promoting improved intestinal absorption of peptides and other materials. These excipients may be used in the present compositions to assist absorption of the SRRI. Additional penetration enhancers include, but are not limited to, polyoxyethylene-9-lauryl ether, polyoxyethylene-20-cetyl ether. For a review of absorption enhancers that find use herein, see B. J. Aungst, “Intestinal Permeation Enhancers,” J. Pharm. Sci., 89 (4), 429 (2000).

Stabilizers and preservatives are generally more important for liquid compositions rather than dry powder compositions. Such substances for oral compositions include the parabens, sorbitol, sodium benzoate, benzoic acid, sorbic acid, potassium sorbate, propionic acid, and combinations thereof. Antioxidants include, but are not limited to, vitamin C, vitamin E, butylated hydroxyanisole (BHA), butylated hydroxytoluene (BHT), and propylgallate. For a review see, I. Himoudy, “Preservatives and their role in pharma and clinical research,” International Journal of Pharma Sciences and Scientific Research, 2:4, 134-151 (2016).

Synergistic Vitamin and/or Mineral Additives

In various embodiments, SSRI composition herein for the treatment of PE may further include at least one vitamin. Vitamins may be included for the purpose of enhancing an individual's sexual performance. For example, any combination of Vitamin B12, Vitamin D, magnesium, and zinc may be included in any of the compositions disclosed herein. Levels may be adjusted such that at least the recommended daily allowance (RDA) of the vitamin and/or mineral is achieved or exceeded.

### Exemplary Compositions, Dosage Forms, Methods of Manufacturing and Methods of Administration

Table 2 sets forth exemplary PE compositions in accordance with the present disclosure Each of the exemplary composition in Table 2 comprise loose powders and each were filled into two-piece hard shell capsules at the fill weights indicated.

TABLE 2
Exemplary SSRI Compositions for PE and Dosages:

| | Compositions | | | | | | | | | | | |
|---|---|---|---|---|---|---|---|---|---|---|---|---|
| Ingredients (wt. %) | 1 | | 2 | | 3 | | 4 | | 5 | | 6 | |
| Dapoxetine-HCl | 21.7 | | -0- | | -0- | | -0- | | -0- | | -0- | |
| Citalopram-HBr | -0- | | 21.7 | | -0- | | -0- | | -0- | | -0- | |
| Escitalopram-oxalate | -0- | | -0- | | 21.7 | | -0- | | -0- | | -0- | |
| Fluoxetine-HCl | -0- | | -0- | | -0- | | 21.7 | | -0- | | -0- | |
| Paroxetine-HCl | -0- | | -0- | | -0- | | -0- | | 21.7 | | -0- | |
| Sertraline-HCl | -0- | | -0- | | -0- | | -0- | | -0- | | 21.7 | |
| Microcrystalline Cellulose | 78.3 | | 78.3 | | 78.3 | | 78.3 | | 78.3 | | 78.3 | |
| Total | 100 | wt. % | 100 | wt. % | 100 | wt. % | 100 | wt. % | 100 | wt. % | 100 | wt. % |
| Capsule Fill | 138 | mg | 138 | mg | 138 | mg | 138 | mg | 138 | mg | 138 | mg |

The microcrystalline cellulose MCC used is preferably Avicel® PH-105 (from DuPont®), or Flocel® 101 (from Medisca®).

The compositions are made according to the following procedure: The SRRI, such as dapoxetine-HCl, is first pulverized to a fine powder, and then mixed together with the MCC in a mortar and pestle or Resonant Acoustic Mixer (RAM). The resulting fine powder is then loaded into individual #3 size capsules at the target fill weight.

### Dosage Regimens

In various embodiments of, methods of treating various aspects of sexual dysfunction are described, including methods for treating premature ejaculation, retarded ejaculation, low sexual desire, sexual aversion, or inhibited orgasm.

In general, a method of treated PE in an individual in need thereof comprises orally administering to the individual a therapeutically effective amount of a PE composition comprising at least one SRRI. In various embodiments, the individual in need thereof has been diagnosed as having premature ejaculation issues, such as manifesting as premature ejaculation, retarded ejaculation, low sexual desire, sexual aversion, or inhibited orgasm.

In various embodiments, a method of treating sexual dysfunction in a male subject in need thereof comprises administering a therapeutically effective amount of a PE composition comprising at least one SRRI as needed and just prior to a sexual encounter. In various aspects, this on-demand or as needed administration replaces the chronic daily dosage regimen currently prescribed for SRRI treatment of PE.

Administration of a PE composition herein is as needed and just prior to an expected sexual encounter. In various examples, just prior to an expected sexual encounter equates to about several minutes to no more than about 3 hours.

PE compositions of the present disclosure are delivered orally, packed into capsules for swallowing. In various embodiments, the PE compositions comprise dry blended loose powders, with the dosage form comprising a hard-shell capsule comprising the dry blended loose powder contained therein. In preferred embodiments, the capsules are fast dissolving, such as comprising vegetarian HPMC, optionally laser perforated to accelerate entrainment of gastric juices upon ingestion.

A therapeutically effective amount of a PE composition is that amount necessary to orally deliver from about 15 mg to about 60 mg total SSRI. In preferred examples, a therapeutically effective amount is about 30 mg of SSRI prior to a sexual encounter. In other examples, an individual may require about 45 mg SSRI, in which case the individual may self-administer one capsule providing about 30 mg SSRI and a second capsule providing about 15 mg SSRI such that the individual self-administers 45 mg SSRI.

In various embodiments, the SSRI is only dapoxetine, the capsule fill weight is about 138 mg and the dosage of dapoxetine in each capsule is about 30 mg. In various examples, an individual in need thereof will self-administer one 138 mg capsule orally no longer than 3 hours prior to an anticipated sexual encounter. The single 138 mg capsule is designed to deliver a single dose of 30 mg dapoxetine.

Compositions for the treatment of premature ejaculation in male subjects and methods thereof are provided. In the detailed description herein, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

All structural, chemical, and functional equivalents to the elements of the above-described various embodiments that are known to those of ordinary skill in the art are expressly incorporated herein by reference and are intended to be encompassed by the present claims. Moreover, it is not necessary for a composition or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112 (f) unless the element is expressly recited using the phrase “means for.” As used herein, the terms “comprises,” “comprising,” or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.

1. A pharmaceutical composition for treatment of a male sexual dysfunction, said composition comprising:
at least one selective serotonin reuptake inhibitor (SRRI);
a carrier; and
optionally, an excipient:
wherein the composition is in the form of a loose dry powder amenable to filling into individual dosage capsules.

2. The pharmaceutical composition of claim 1, wherein the selective serotonin reuptake inhibitor (SRRI) is selected from the group consisting of dapoxetine, citalopram, escitalopram, fluoxetine, paroxetine, sertraline, pharmaceutically acceptable salts thereof, and mixtures thereof.

3. A pharmaceutical dosage form for oral administration comprising:
a hard-shell capsule; and
a composition according to claim 1 enclosed therein.

4. The pharmaceutical composition of claim 1, wherein the carrier comprises microcrystalline cellulose.

5. The pharmaceutical composition of claim 1, wherein the optional excipient is at least one of a disintegrant, a colorant, a flavoring agent, a sweetener, a preservative, and a stabilizer.

6. The pharmaceutical composition of claim 1 consisting essentially of dapoxetine-hydrochloride and microcrystalline cellulose.

7. A single dose pharmaceutical capsule consisting essentially of:
from about 100 mg to about 300 mg of a composition consisting essentially of from about 15 wt. % to about 25 wt. % dapoxetine-hydrochloride and from about 75 wt. % to about 85 wt. % microcrystalline cellulose, based on the total weight of the composition; and
a hard-shell capsule enclosing said composition.

8. The single dose pharmaceutical capsule of claim 7, wherein the capsule comprises gelatin or vegetarian hydroxypropyl methylcellulose (HPMC), and wherein the capsule is optionally laser-perforated with a plurality of holes for water ingress.

9. The single dose pharmaceutical capsule of claim 7, wherein each capsule provides about 15 mg to about 60 mg dapoxetine.

10. A method of treating a sexual dysfunction in a male individual, the method comprising orally administering to the individual in need thereof a therapeutically effective amount of a loose powder composition packaged in an immediate delivery capsule dosage form, said composition comprising at least one selective serotonin reuptake inhibitor (SRRI); a carrier; and optionally, an excipient, wherein the SRRI is selected from the group consisting of dapoxetine, citalopram, escitalopram, fluoxetine, paroxetine, sertraline, pharmaceutically acceptable salts thereof, and mixtures thereof.

11. The method of claim 10, wherein the sexual dysfunction is characterized by premature ejaculation, retarded ejaculation, low sexual desire, sexual aversion, or inhibited orgasm.

12. The method of claim 10, wherein the therapeutically effective amount comprises orally administering from about 15 mg to about 60 mg of the at least one SRRI as needed and within 3-hours prior to an expected sexual encounter.

13. The method of claim 10, wherein the at least one SRRI is dapoxetine and the carrier is microcrystalline cellulose.

14. The method of claim 13, wherein said therapeutically effective amount comprises about 138 mg of said loose powder composition.

15. The method of claim 9, wherein said therapeutically effective amount comprises from about 15 mg to about 60 mg of dapoxetine.
